(19) United States
(12) Patent Application Publication
(10) Pub. No.: US 2024/0115620 A1
(43) Pub. Date: Apr. 11, 2024

(54) PREPARATION AND THERAPEUTIC USE OF 3D-CULTURED FIBROBLASTS

(71) Applicant: FIBROBIOLOGICS, Webster, TX (US)

(72) Inventors: Bin JIANG, Webster, TX (US); Hamid KHOJA, Webster, TX (US); Pete O'HEERON, Houston, TX (US)

(21) Appl. No.: 18/483,264

(22) Filed: Oct. 9, 2023

Related U.S. Application Data
(60) Provisional application No. 63/378,796, filed on Oct. 7, 2022.

(51) Int. Cl. A61K 35/33; C12N 5/077

(52) U.S. Cl. CPC A61K 35/33; C12N 5/0656; C12N 2500/32; C12N 2500/34; C12N 2509/00; C12N 2513/00

(57) ABSTRACT

The present disclosure provides methods, compositions, and systems for producing and using organoids comprising fibroblasts. The organoids can be generated and placed in a particular storage matrix which allows for sufficient conditions for transport and reduces damage to cells in the organoids compared to known media. The organoids may be utilized for extended release of the fibroblasts for cell therapy and also for drug delivery at a target site following administration to an individual in need thereof.

This application claims priority to U.S. Provisional Patent Application Ser. No. 63/378,796, filed Oct. 7, 2022, which is incorporated by reference herein in its entirety.

### TECHNICAL FIELD

Embodiments of the disclosure concern at least the fields of cell biology, molecular biology, immunology, and medicine.

### BACKGROUND

Beginning at the embryonic phase of human development, fibroblasts play a crucial role in every aspect of human physiology, anatomy, and immunology.[1]

Fibroblasts can be isolated from a variety of tissues. This extraction method relies on specific equipment in a high-standard laboratory (ultra-clean bench, constant temperature humidity incubator, and various high-purity gases and incubators) for preparation, maintenance, and quality control. Ordinary medical institutions do not meet these conditions. Therefore, fibroblast products for cell therapy must transport over a long distance between experimental and medical institutions across a specific geographic range[2]. Under the ambient conditions, the cells will rupture their organelles, and the nuclear damage will eventually lead to apoptosis; the cell debris can cause severe inflammation post-transplantation[3]. Currently, there are two types of cell transportation: short-distance and long-distance transportation. Short-distance transportation is usually limited to 24 hours[4]. If more than 24 hours, the cells can be covered in the culture flask with growth media. A 37° C. thermostatic suitcase is transported close to the body at room temperature, but the amount of cells transported in this way is limited and can easily cause cell damage. Even if the cells arrive at the destination, it takes 48 hours for them to recover. Technically, this method consumes a lot of expensive culture media, and it is a potential public safety hazard to carry liquids. Transportation methods such as planes and high-speed trains are dangerous and inefficient, so they can only be used to transfer a small number of living cell lines, etc. However, small-volume cell products are unsuitable for cell therapy, which requires up to a billion cells per case. For long-distance transportation of a large number of cells, dry ice delivery is widely used [5, 6]. The cells are frozen in a cryovial, placed in solid dry ice, transported to the destination laboratory/hospital, thawed, and used after 4-7 days of cell recovery through culturing on-site. Although this method can transport many cells at one time, it requires the addition of dozens of kilograms of dry ice every 12-24 hours. Each stop along the delivery chain must contact the local supplier to provide dry ice, which is very expensive. Dry ice is also hard to take on a plane. High-speed rail and other means of transportation require complicated procedures and certification materials, inevitably increasing the cost. If the dry ice is consumed en route and cannot be replenished, the cells may undergo freeze-thaw cycles, which will cause irreversible damage to the cells. Therefore, developing a method to efficiently and safely transport cells at room temperature is crucial to realizing cell-applied therapy.

### BRIEF SUMMARY

Embodiments of the disclosure encompass compositions, methods, and systems for the efficient preparation, culturing, maintenance, preservation, and/or transportation of fibroblasts that are not in a state of a plurality of single cells. Embodiments of the disclosure encompass compositions, methods, and systems for the efficient preparation, culturing, maintenance, preservation, and/or transportation of fibroblasts that exist as a cluster, such as a cluster of at least 2, 10, 50, 100, 500, 1000, 2000, 500, 7500, 10,000, 25,000, 50,000, 75,000, 100,000, and so forth number of cells. In specific embodiments, the cluster of cells (which may be referred to as an organoid) exhibits a particular shape, such as a sphere or a shape that is sphere-like, a cube or a shape that is cube-like, a pyramid or a shape that is pyramid-like, a star or a shape that is star-like, and so forth. In other embodiments, the organoid has no particular geometric form. In specific embodiments, the cluster of fibroblast cells comprises a 3D fibroblast spheroid.

The present disclosure encompasses 3D fibroblast organoids in which at least about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the cells in the organoids are fibroblasts. In specific cases, the majority of cells in the 3D fibroblast organoids are fibroblasts. The 3D fibroblast organoids may comprise about 50-100, 50-90, 50-80, 50-70, 50-60, 60-100, 60-90, 60-80, 60-70, 70-100, 70-90, 70-80, 80-100, 80-90, or 90-100% fibroblasts, including any value or range derivable therein. The 3D fibroblast spheroid may include a combination of one or more types of cells other than fibroblasts, such as stem cells, epithelial cells, keratinocytes, neurons, and/or immune cells of any kind.

Any of the fibroblast organoids, including spheroids, can be used in disease modeling, clinical training, research, and/or as therapeutic uses for humans and animals, including mammals of any kind. In particular embodiments, the disclosure concerns the interaction of a first type of cells with a second type of cells and/or certain agent(s) and includes modification(s) to the first and/or second type of cells as a result of the interaction. In specific embodiments, the first type of cells are fibroblasts, and the second type of cells are immune cells of any kind (e.g., T cells, NK cells, NKT cells, macrophages, B cells, dendritic cells, a mixture thereof, etc.), stem cells of any kind, or a combination thereof. In addition, in specific embodiments, the disclosure includes compositions, methods, and systems in which fibroblasts are modified upon exposure to certain cells and/or one or a combination of certain conditions, environments, and/or agent(s). Specific agents that may modify the fibroblasts include one or more of nucleic acids, cytokines, chemokines, growth factors, and/or exosomes prior to and/or during the preparation, culturing, maintenance, and or preservation of the single cell fibroblast and/or following generation of the 3D fibroblast organoids, including 3D fibroblast spheroids. In addition, in specific embodiments, the disclosure provides introduction of these fibroblast spheroids, or materials derived from the fibroblast spheroids, such as for use as an extended time release mechanism of the fibroblasts and/or fibroblast-derived materials in vivo. In certain embodiments, such materials can be utilized for clinical applications of any kind, including the following: tissue regeneration, immune modulation, tissue repair, wound healing, and/or as a targeted tool of cell migration to specific cell types or tissues. In cases wherein the fibroblast organoids are utilized as a targeted tool of cell migration to specific cell types or tissues, in specific embodiments this occurs through activated or engineered surface markers, nucleic acid modification, and/or expression or excretion of one or more chemokines, one or more cytokines, exosomes, and/or one or more growth factors.

Embodiments of the disclosure provide an in vitro or in vivo method of producing cell structures of any kind, or materials derived therefrom, including (1) activated or unactivated fibroblast spheroids; (2) fibroblast spheroid-derived materials from activated or unactivated spheroid fibroblasts; (3) activated or unactivated immune cells, and/or (4) fibroblast derivatives. Such compositions may be utilized for any suitable application, including at least for introduction in vivo (in some cases, subsequent to their in vitro or ex vivo generation) to (1) initiate or maintain extended time release immune modulation, (2) initiate or maintain extended time release of activated or inactivated fibroblasts and/or fibroblast-derived material, and/or (3) initiate or maintain extended time release migration and proliferation to a targeted tissue or organ for the purpose of repair, regeneration and/or reversal of involution, as examples. In specific embodiments, the composition(s) that are therapeutic are the spheroids or cells released therefrom themselves, whereas in additional or alternative embodiments the composition(s) that are therapeutic are materials derived from cells of the spheroid that are released from the spheroids or cells released therefrom. In situations where the therapy is, or originates from, cells released from the organoids over time, the timing of release may have a controllable or predictable rate of release of the cells or the moieties released from the cells. Such a control of time release may or may be the direct or indirect result of how the organoids were prepared. The cells within the spheroids may be allogeneic, autologous, or xenogeneic with respect to a recipient individual.

The fibroblast cells may be activated, such as having activated or engineered surface markers, nucleic acid modification, and/or expression or excretion of one or more chemokines, cytokines, exosomes, and/or growth factors.

In various embodiments, the disclosure encompasses the preparation, culturing, maintenance, preservation, transportation, and/or utilization of organoid fibroblasts, including fibroblast spheroids and/or fibroblast spheroids comprising one or more types of other cells and/or cell components for which the structures can be used for any reason, including at least as extended time release of fibroblast cells and or fibroblast-derived materials for use in clinical applications.

One example of a clinical application for the structures comprises methods for preserving and/or transporting fibroblasts at a particular desired temperature (e.g., about 0-37° C.), including utilizing a unique matrix to ensure their viability and biological activity. The methods of the present disclosure are different from any previous methods. In one embodiment, there is utilization of the characteristics of fibroblast spheroids formation to reduce their proliferation and metabolic rate and to preserve the vitality and biological activity fibroblasts and/or fibroblasts, such as, in some cases, in combination with one or more other type of cells, including for up to at least about 21 or more days, e.g., under ambient conditions.

Another example of a clinical application for the structures includes using a unique liquid storage matrix, which in particular embodiments has no biological toxicity, which can be easily applied to the single cells and/or formed structures and that meet the requirements of high-density and large-scale storage and transportation of cells. In particular embodiments, cell products do not require subsequent separation and purification steps prior to use, and the entire storage and transportation process does not need to maintain specific temperatures and humidity. As a result, the cell products can directly be used for biological and medical scientific research, cell therapy for immune diseases and degenerative diseases, cell transplantation for tissue and organ damage, and to satisfy the requirements for drug screening, with immeasurable scientific and socioeconomic benefits.

Embodiments of the disclosure utilize the ability of fibroblasts to form organoids, such as spheroids, under particular suspension culture conditions, in which case fibroblasts can spontaneously aggregate to form compacted organoids. In particular embodiments in the cell spheroids, the fibroblast cells naturally reduce their proliferation and metabolism rates to reduce oxygen consumption and nutrient consumption resulting from cell contact inhibition.

The present disclosure provides a unique liquid culture storage medium, or matrix, which can ensure that the organoids have the most basic energy, nutrition maintenance needs, and/or suitable pH for cell survival and to reduce the shear stress damage inherent in cell use and transport, such as caused by sudden movements during transportation. Moreover, the methods of the present disclosure enable cells to meet long-distance transportation (e.g., transportation for over 24 hours) needs under normal temperature conditions, such as and/or for the cell products only to be stored in plastic, glass, metal, and/or rubber containers. In some embodiments involving large-scale transportation of cells with a high density (e.g., transportation of at least 1 million to 100 billion cells), the method does not require any specific temperature and gas maintenance equipment, although certain embodiments may include such for cryopreservation and/or transportation.

In particular embodiments, after the cells arrive at the intended destination (e.g., university labs, research institutes, medical facilities of any kind, including hospitals, etc.), the matrix can be removed by any suitable method, including at least centrifugation. As demonstrated herein, the collected cells have a clinically beneficial survival rate with complete biological function after at least seven days of storage under ambient conditions, rendering them useful for assays of any kind and/or treatment of any kind.

In various embodiments, the method for fibroblast spheroid formation may utilize U/V bottom type ultra-low attachment culture plates, a hanging drop method, applying the fibroblasts to a hydrophilic region of a substrate comprising a substantially hydrophobic surface, and/or forming a core-shell structure within a pipette tip (e.g., (1) making the core spheroid in a single pipette tip and culturing for 48 hours; (2) creating a second layer on the spheroids with cells in the same pipette tip and culturing for 48 hours, repeating as desired to add additional layers of cells and/or cell types to the spheroid; and (3) after the cells spheroids form in the pipette tip for 96 hours, collecting the cell spheroids from the pipette tip by ejecting the liquid containing the spheroid from inside the pipette tip, removing the liquid matrix using centrifugation or filtration, and collecting the cell spheroids). The methods may generate cell spheroids of different sizes according to the adjustment of the cell density of cells. In particular embodiments, the sphere size can be between about 50-500, 50-400, 50-300, 50-200, 50-100, 100-500, 100-400, 100-300, 100-200, 200-500, 200-400, 200-300, 300-500, 300-400, or 400-500 microns, including any value or range derivable therein. The sphere size may be about 50, 75, 100, 125, 150, 175, 200, 225, 250, 275, 300, 325, 350, 375, 400, 425, 450, 475, or 500 microns, including any value or range derivable therein. In some embodiments, ultra-low attachment culture plates or glass culture flasks can be directly utilized to spread fibroblast cells into containers or culture dishes at a high density so they spontaneously form spheroids of different sizes.

In particular embodiments, for transport or preservation means, one or more bio-safe agents (i.e., tackifier) may be included in the liquid storage matrix, which can increase the viscosity of the medium and preserve the fibroblast spheroids in the liquid storage matrix. In a particular embodiment, the liquid storage matrix comprises a base cell culture medium that comprises essential nutrients and an acid-base balance system, except phenol red. In specific embodiments the base cell culture medium comprises a low-sugar base medium, human serum, non-essential amino acids, and/or L-glutamine. The tackifier for increasing the viscosity of the medium may be a food-grade additive, such as Methylcellulose, Agar, Guar gum, Xanthan gum, Pectin, Collagen, or Gelatin, and in some cases the concentration is about 0.2-0.5, 0.2-0.4, 0.2-0.3, 0.3-0.5, 0.3-0.4, or 0.4-0.5%, including any value or range derivable therein. The concentration of the agent may be 0, about 0.1%, about 0.2%, 0.3%, 0.4%, 0.5%, 0.6%, 0.7%, 0.8%, 0.9%, 1%, 2%, 3%, 4%, or 5%, including any value or range derivable therein. The medium may contain other materials and conditions for culture success. The tackifier does not produce biological toxicity, can be metabolized, has no residue, and is safe for cells, in specific embodiments. In particular embodiments, the fibroblast organoids can survive up to at least 1, 2, 3, 4, 5, 6, 7, 8, 9, 10, 11, 12, 13, 14, 15, 16, 17, 18, 19, 20, or 21, including any value or range derivable therein, days without being damaged. In specific embodiments, the organoids can be transported anywhere within about 1-21, 1-20, 1-15, 1-10, 1-5, 1-2, 2-21, 2-20, 2-15, 2-10, 2-5, 5-21, 5-20, 5-10, 10-21, 10-20, 10-15, 15-21, 15-20, or 20-21, including any value or range derivable therein, days and avoid shear stress damage caused during transport.

In various embodiments of the disclosure, there is clinical use of the fibroblast spheroids, e.g., for the purpose of an extended time release for any therapeutic application, including sustained immune modulation capability, such as in autoimmune disorders.

In various embodiments of the disclosure, there is clinical use of the fibroblast spheroids, e.g., for the purpose of an extended time release for sustained release of one or more or a combination of fibroblast-derived growth factors.

In various embodiments of the disclosure there is clinical use of the fibroblast spheroids for the purpose of clinical or biological research.

Compositions and methods of the present disclosure allow for increasing the survivability of the fibroblasts for use of any kind in vivo, during and after delivery, and increasing the survivability of spheroids to reach damaged tissue regions for sustained proliferation and repair of damaged tissue and organs or for reversing organ involution.

In specific embodiments, the disclosure concerns compositions, methods, and systems in which certain cells are modified upon exposure to fibroblasts and/or certain agent(s) excreted from fibroblast spheroids or contained within the fibroblast spheroids or fibroblast cells. In particular embodiments, the interaction of fibroblasts with one or more other types of cells (and optionally, that interaction also includes one or more certain environments and/or agent(s)) results in modification of the fibroblasts and/or the other type of cells such that become involved in immune modulation, tissue repair, and reversal of tissue and organ involution. In specific embodiments, the other types of cells include at least recruited immune cells and local stem cells.

In certain embodiments, methods include the introduction of a live single cell suspension or live 3D fibroblast spheroids, inactivated fibroblasts, and/or fibroblast-derived materials to a targeted tissue or organ for the modification of fibroblasts and other cells found in the targeted tissue or organ and/or surrounding tissue. Such activity in specific embodiments at least stimulates tissue and/or organ repair or reversal of the involution process. In certain aspects, there is exposure of fibroblasts and/or one or more agents produced from modifications to one or more types of cells present in the injury site. In specific embodiments, one or more agents are included in the exposure and may or may not be exogenously provided, such as in other cases where they are endogenous to an environment and/or cell and/or tissue.

In specific embodiments, methods of the disclosure occur ex vivo, such as in a culture. In particular cases, the methods occur by the hand of man and do not encompass ordinary or random occurrences in a body. The methods of the disclosure are non-natural, in particular aspects. In specific embodiments, the concentrations of cells or cell-derived materials used in a method of exposing one type of cells to another type of cells do not occur in nature and do not happen randomly in nature. In specific embodiments, the concentration of one or more agents used in a method of exposing one or more agents to one or more types of cells does not occur in nature and does not happen randomly in nature. The modification of any types of cells encompassed by the disclosure that occurs ex vivo or in vitro does not occur in vivo naturally in the same manner. In such embodiment, tissue biopsy from the donor is used to isolate, characterize, if necessary, activate, expand, and reintroduce back into the donor a combination of the cells in an organoid structure for the purpose of immune modulation, tissue repair, tissue regeneration, and/or reversal of tissue involution.

The disclosure in particular embodiments encompasses therapeutic uses of cells, including fibroblasts of any kind (including organ fibroblasts), epithelial cells, immune cells, neutrophils, macrophages, keratinocytes, vasal epithelial cells, myofibroblasts, and mixtures thereof. In at least some cases, the fibroblasts or fibroblast-derived materials have been modified prior to their exposure introduction to the targeted tissue or organ, such as chemically, virally, physically, or epigenetically activated, or exposed to conditions not normally found in the body. In other cases, immune cells such as neutrophils, macrophages, tissue-specific fibroblasts, keratinocytes, vasal epithelial cells, myofibroblasts, or their derivatives, have been modified, such as activated, prior to their exposure to the fibroblasts.

Embodiments of the disclosure provide means of utilizing fibroblasts as allogeneic, autologous (or xenogeneic or syngeneic) therapeutic cells through modification of culture conditions. In one embodiment of the disclosure, fibroblasts are extracted from sources with lower immunogenicity (e.g., placental fibroblasts, omental tissue-derived fibroblasts, cord blood-derived fibroblasts, etc.), or other sources which may produce cells which can be used for therapeutic or clinical research purposes.

In one embodiment of the disclosure, fibroblasts are cultured as spheroids in vitro to preserve fibroblasts' viability and proliferative ability. The disclosure provides for the modification of known culture techniques to decrease the recognition of fibroblasts by the recipient's immune system. In one embodiment, fibroblast spheroids are cultured in conditions that lack xenogeneic components, such as a xenogeneic-free medium. In some cases, for example, the media is free of fetal calf serum. In specific embodiments, the disclosure encompasses the substitution of fetal calf serum with one or more other agents, such as those that facilitate the reduction of immunogenicity of fibroblast spheroids, for example, human platelet-rich plasma, platelet lysate, umbilical cord blood serum, autologous serum, and/or one or more defined cytokines, such as one or a combination of fibroblast growth factor, epidermal growth factor, leukemia inhibitory factor, insulin-like growth factor, angiopoietin, and vascular endothelial growth factor, or other agents.

In one embodiment of the disclosure, effective amounts of fibroblast spheroids, as prepared in methods encompassed by the disclosure, are administered to an individual for therapy or prevention of one or more medical conditions. In specific embodiments, fibroblast spheroids and/or fibroblast spheroid-derived materials are administered to improve immune response, wound repair, cell differentiation, and/or tissue remodeling in the recipient individual.

Embodiments of the disclosure provide methods for co-administration of universal donor fibroblast spheroids (whether or not the fibroblasts are gene-modified xenogenetically, allogeneically, or autologously modified) with one or more agents, such that individually or in combination stimulate and maintain a desired immune response, tissue repair, and/or tissue regeneration. In a specific embodiment of the disclosure, methods are provided for co-administration of universal donor fibroblast spheroids and/or fibroblast spheroid-derived materials with one or a combination of chemokines and/or cytokines and/or growth factors. In one embodiment of the disclosure, universal donor fibroblast spheroids derived from fibroblast spheroids that have been treated under conditions to reduce immunogenicity are utilized to stimulate fibroblast growth factors, such that are required for tissue repair, tissue regeneration, and/or reversal of organ involution.

Embodiments of the disclosure provide methods of reducing the immunogenicity of particular types of fibroblast spheroids. Fibroblast spheroids may be derived from various tissues or organs, including but not limited to skin, heart, blood vessels, bone marrow, skeletal muscle, liver, pancreas, brain, foreskin, which can be obtained by biopsy (where appropriate), or upon autopsy. In some aspects, the cells comprise fibroblast spheroids, which can be from a fetal origin, neonatal origin, adult origin, or a combination thereof.

Fibroblast spheroids for use in any methods of the disclosure may be exposed to certain medium component(s) in specific embodiments.

In embodiments, there is a method of producing fibroblast organoids, comprising the step of producing a plurality of fibroblast organoids; and suspending the organoids in a medium comprising low-glucose medium; from about 0 to 20% human serum; about 0 to 5% non-essential amino acids; and/or about 0 to 5% L-glutamine; and optionally one or more tackifiers. In specific embodiments, the amount of human serum is about 0-20, 0-15, 0-10, 0-5, 1-20, 1-15, 1-10, 1-5, 5-20, 5-15, 5-10, 10-20, 10-15, or 15-20%, including any value or range derivable therein. The amount of serum may be about 0, 1, 2, 3, 4, 5, 6, 7, 8, 9, 10, 11, 12, 13, 14, 15, 16, 17, 18, 19, or 20%, including any value or range derivable therein. In specific embodiments, the amount of non-essential amino acids is about 0-5, 0-4, 0-3, 0-2, 0-1, 1-5, 1-4, 1-3, 1-2, 2-5, 2-4, 2-3, 3-5, 3-4, or 4-5%, including any value or range derivable therein. In some cases, the amount of non-essential amino acids is about 0, 1, 2, 3, 4, or 5% non-essential amino acids, including any value or range derivable therein. In specific embodiments, the non-essential amino acids include glycine, L-alanine, L-aspargine monohydrate, L-aspartic acid, L-glutamic acid, L-proline, L-serine, L-histidine, Isoleucine, L-lysine hydrochloride, L-serine, L-tryptophan, and/or L-valine. The amount of L-glutamine may be about 0-5, 0-4, 0-3, 0-2, 0-1, 1-5, 1-4, 1-3, 1-2, 2-5, 2-4, 2-3, 3-5, 3-4, or 4-5% L-glutamine, including about 0, 1, 2, 3, 4, or 5% L-glutamine, including any value or range derivable therein. In certain embodiments, the amount of tackifier is about 0-5, 0-4, 0-3, 0-2, 0-1, 1-5, 1-4, 1-3, 1-2, 2-5, 2-4, 2-3, 3-5, 3-4, or 4-5%, such as being about 0, 1, 2, 3, 4, or 5%, including any value or range derivable therein. Examples of tackifiers include Methylcellulose, Agar, Guar gum, Xanthan gum, Pectin, Collagen, and/or Gelatin.

In specific embodiments, the produced size of the organoids is about 50-500, 50-400, 50-300, 50-200, 50-100, 100-500, 100-400, 100-300, 100-200, 200-500, 200-400, 200-300, 300-500, 300-400, or 400-500 microns, including any value or range derivable therein. The produced size of the organoids may be about 50, 75, 100, 125, 150, 175, 200, 225, 250, 275, 300, 325, 350, 375, 400, 425, 450, 475, or 500 microns, including any value or range derivable therein. In specific embodiments, the organoids are produced by one of the following: (a) using a U/V bottom type ultra-low attachment culture plate; (b) the hanging drop method; (c) applying the cells to a hydrophilic region of a substrate comprising a substantially hydrophobic surface; and/or (d) forming a core-shell structure within a pipette tip (e.g., (1) making the core spheroid in a single pipette tip and culturing for 48 hours; (2) creating a second layer on the spheroids with cells in the same pipette tip and culturing for 48 hours, repeating as desired to add additional layers of cells and/or cell types to the spheroid; and (3) after the cells spheroids form in the pipette tip for 96 hours, collecting the cell spheroids from the pipette tip by ejecting the liquid containing the spheroid from inside the pipette tip, removing the liquid matrix using centrifugation or filtration, and collecting the cell spheroids). In specific embodiments, the number of cells in the organoids are about 10³, 10⁴, 10⁵, 10⁶, 10⁷, 10⁸, 10⁹, or 10¹⁰ cells per organoid. The fibroblast organoids may be produced upon culture in an incubator at about 37° C. for about 24-48 hours, wherein the incubator comprises about 5% carbon dioxide and greater than about 80% humidity. In specific cases, the fibroblast organoids are produced upon culture for about 24-48, 24-44, 24-40, 24-36, 24-30, 24-28, 28-48, 28-44, 28-40, 28-36, 28-30, 30-48, 30-44, 30-40, 30-36, 36-48, 36-44, 36-40, 40-48, 40-44, or 44-48 hours, including any value or range derivable therein. The fibroblast organoids may be produced upon culture for about 24, 25, 26, 27, 28, 29, 30, 31, 32, 33, 34, 35, 36, 37, 38, 39, 40, 41, 42, 43, 44, 45, 46, 47, or 48 hours, including any value or range derivable therein. The fibroblast organoids may be produced upon culture at a temperature of about 20, 21, 22, 23, 24, 25, 26, 27, 28, 29, 30, 31, 32, 33, 34, 35, 36, or 37° C., including any value or range derivable therein. In specific embodiments, the fibroblast organoids are produced upon culture at a temperature of about 20-37, 20-35, 20-30, 20-27, 20-25, 20-22, 22-37, 22-35, 22-33, 22-30, 22-27, 22-25, 25-37, 25-35, 25-33, 25-30, 25-27, 27-37, 27-35, 27-33, 27-30, 30-37, 30-35, 30-33, 33-37, 33-35, or 35-37° C., including any value or range derivable therein.

In specific embodiments, the fibroblast organoids are produced upon culture in carbon dioxide at about 3, 4, 5, 6, 7, or 8%, such as produced upon culture in carbon dioxide at about 3-8, 3-7, 3-6, 3-5, 3-4, 4-8, 4-7, 4-6, 4-5, 5-8, 5-7, 5-6, 6-8, 6-7, or 7-8%, including any value or range derivable therein. The fibroblast organoids may be produced upon culture in about 65, 70, 75, 80, 85, or 90% humidity, including produced upon culture in about 65-90, 65-85, 65-80, 65-75, 65-70, 70-90, 70-85, 70-80, 70-75, 75-90, 75-85, 75-80, 80-90, 80-85, or 85-90% humidity, including any value or range derivable therein. The producing step may comprise modification of one or more parameters to control the size of the produced organoid. In specific embodiments, the one or more parameters that are modified comprise the concentration of fibroblast cells; the type and/or amount of base media; the type and/or amount of sugar in the media; the type and/or amount of serum; the type and/or non-essential amino acids; the amount of L-glutamine; the amount of carbon dioxide in an incubator comprising the cells; the temperature in the incubator; the duration of time of production of the spheroid; or a combination thereof.

In certain embodiments, the organoids are placed in a container, such as plastic, metal, glass, rubber, and so on. The organoids may be stored following production, including at ambient temperature, in some embodiments. In some cases, the organoids are stored at about 2-37° C. The organoids may be cryogenically frozen.

In some embodiments, the organoids are transported, for example within about 0-50 days, including within 0-50, 0-15, 0-10, 0-5, 1-20, 1-15, 1-10, 1-5, 5-20, 5-15, 5-10, 10-20, 10-15, 15-20, 20-25, or 25-30 days, including any value or range derivable therein. The organoids may be transported at ambient temperature, including at about 2-37° C. Following transportation, the organoids are removed from the media, such as by centrifugation, gravity, filtration, microfluidic cassettes, mechanical interaction, chemical interaction, or a combination thereof. Following transportation, at least some of the cells are dissociated from the organoid. The cells maybe dissociated from the organoid upon being exposed to an effective amount of one or more proteases, such as trypsin, and the amount of trypsin may be about 0.1-0.3% trypsin, including about 0.25%.

In various embodiments, the organoids are used within 0-21 days of any production method encompassed herein. In some embodiments, the organoids are subject to one or more research assays or are used for therapy within 0-20, 0-15, 0-10, 0-5, 1-20, 1-15, 1-10, 1-5, 5-20, 5-15, 5-10, 10-20, 10-15, or 15-20 days, including any value or range derivable therein, of any production method encompassed herein. In some embodiments, the organoids are subject to one or more research assays or are used for therapy within 0, 1, 2, 3, 4, 5, 6, 7, 8, 9, 10, 11, 12, 13, 14, 15, 16, 17, 18, 19, or 20 days, including any value or range derivable therein, of any production method encompassed herein. The organoids may be used in one or more research assays as an alternative to an animal model, for example for pre-clinical drug testing or drug development. In some embodiments, the research assay is for using the organoids to study organ function. In specific embodiments, the research assay is for using the organoids to satisfy requirements for drug screening. In specific embodiments, any method encompassed herein may further comprise the step of exposing the organoids to one or more chemicals or one or more drugs.

In particular embodiments, any method encompassed herein may further comprise the step of administering a therapeutically effective amount of the organoids to an individual in need thereof. In specific embodiments, the individual may have an autoimmune, chronic, degenerative, genetic, infectious, and/or other disorder or disease, and/or is in need of immune modulation, tissue repair, tissue regeneration, organ repair, and/or organ regeneration. The autoimmune disorder and/or disease may be multiple sclerosis, eczema, and/or psoriasis. The infectious disorder and/or disease may be viral, bacterial, fungal, and/or protozoan. The genetic disorder and/or disease may be cancer. The organoids may be administered locally or systemically. They may be administered by injection, infusion, spray, and/or inclusion in a 3D matrix. In certain embodiments, the organoids are administered to the individual once or multiple times. In specific cases, when there are multiple administrations, the duration in time between the administrations may be within 1, 2, 3, 4, 5, 6, 7, 8, 9, 10, 11, or 12 hours, or within 1, 2, 3, 4, 5, 6, or 7 days, or within 1, 2, 3, or 4 weeks, or within 1, 2, 3, 4, 5, 6, 7, 8, 9, 10, 11, or 12 months, or within 1, 2, 3, 4, 5, or more years, including any value or range derivable therein.

In various embodiments, fibroblasts in the organoids are dissociated from the organoids following administration, and they may be dissociated from the organoids as an extended release. The release may be over any duration, including over the course of about one hour to about 15 days, as examples. In particular embodiments, the fibroblasts migrate to a specific tissue or organ, including one in need of tissue repair and/or for eliciting an immune response at the specific tissue or organ. The specific organ may be the heart, liver, lungs, stomach, spleen, gall bladder, kidney, brain, bladder, and/or intestines. The specific tissue may be connective tissue, epithelial tissue, muscle tissue, and/or nervous tissue.

In various embodiments, the fibroblasts in the organoids are activated, and they may be activated by one or more chemical agents, RNA, micro RNA, RNAi, DNA, viral nucleic acid, and/or exosomes. In some embodiments, the fibroblasts comprise one or more therapeutic agents for release from the fibroblasts. Any method encompassed herein may further comprise the step of modifying the fibroblasts to comprise the one or more therapeutic agents. Examples of a therapeutic agent is a protein, nucleic acid, exosomes, growth factors, micro RNA, RNAi, mRNA, or a combination thereof.

Embodiments of the disclosure include compositions comprising organoids produced by any method encompassed herein. The organoids may be comprised in a liquid storage matrix. The composition may comprise organoids that are at ambient temperature. The organoids may be frozen. The composition may comprise organoids in a container, including one that is plastic, glass, metal, or rubber. In specific embodiments, the composition comprises organoids that are in a liquid storage matrix comprising low-glucose medium; from about 0 to 20% human serum; about 0 to 5% non-essential amino acids; and/or about 0 to 5% L-glutamine; and optionally one or more tackifiers.

The foregoing has outlined rather broadly the features and technical advantages of the present invention so that the detailed description of the invention that follows may be better understood. Additional features and advantages of the invention will be described hereinafter, which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF DRAWINGS

FIG. 1. Mouse fibroblast cells can be formed into spheroids via the hanging drop method.

FIG. 2. Mouse fibroblast cells maintain 100% survival following storage at ambient conditions for one week.

FIG. 3. Mouse fibroblast cells can undergo ambient condition storage for one week and maintain normal fibroblast morphology, proliferation, and migration capability.

FIG. 4. Human skin fibroblast (e.g., human dermal fibroblast, e.g., HDF) cells can maintain 100% survival following storage at ambient conditions for one week.

FIG. 5. Human skin fibroblast cells in spheroids can maintain 95% survival following storage at ambient conditions for two weeks. The HDF spheroids were tested for survival rate with AO/PI. There is no significant difference between the HDF that underwent 2 weeks of storage at 4° C. and the control with no storage.

FIG. 6. Human skin fibroblast cells in spheroids can maintain 89% survival following storage at 4° C. for four weeks, as detected with AO/PI staining. The preservation medium can be 1×DPBS, 10% FBS, and 10% DMEM/F12.

FIG. 7. Human skin fibroblast cells in spheroids can undergo storage for 2 and 4 weeks at 4° C., then be replated for 5 days, and maintain their migration and proliferation capabilities when reattached to the cell culture plates.

FIG. 8. Human skin fibroblast cells can express both the mesenchymal and HDF-specific biomarkers following storage for 4 weeks at 4° C. Cryosections taken after 4 weeks show that fibroblast spheroids have a high-density layer in the peripheral and low density in the center of the spheroid. Staining of the cryosections demonstrates that HDF spheroids highly express fibroblast-specific markers (i.e., Actin, S00A4, and Vimentin) and partial mesenchymal markers (i.e., CD44 and CD90).

### DETAILED DESCRIPTION

### I. Examples of Definitions

In keeping with long-standing patent law conventions, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight, or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicate the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated immune cells” refers to immune cells treated with one or more stimuli capable of inducing one or more alterations in the cell: metabolic, immunological, epigenetic, growth factor secreting, surface marker expression, and production and excretion of microvesicles.

The term “administered” or “administering,” as used herein, refers to any method of providing a composition to an individual such that the composition has its intended effect on the patient. For example, one method of administering is by an indirect mechanism using a medical device such as, but not limited to, a catheter, applicator gun, syringe, gel matrix, and a 3D matrix containing one or more cell types, cell-derived products, and or growth factors and or antibiotics, etc. A second exemplary method of administering is by a direct mechanism such as local tissue administration, oral ingestion, transdermal patch, topical, inhalation, suppository, etc.

As used herein, “allogeneic” refers to tissues or cells from another body that, in a natural setting, are immunologically incompatible or capable of being immunologically incompatible, although from one or more individuals of the same species.

As used herein, “autologous” refers to tissues or cells that are derived or transferred from the same individual's body (i.e., autologous blood donation; an autologous bone marrow transplant).

As used herein, “agent” refers to nucleic acids, cytokines, chemokines, transcription factors, epigenetics factors, growth factors, hormones, or a combination thereof, including whole cell lysate.

As used herein, “xenogeneic” refers to tissues or cells from a species different from the patient.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO 2 at various concentrations. Culture conditions may vary widely for each cell type, though, and variation of conditions for a particular cell type can result in different phenotypes being expressed. The most commonly varied factor in culture systems is the growth medium and oxygen concentration during culturing. Growth media can vary in concentration of nutrients, growth factors, and the presence of other components. The growth factors used to supplement media are often derived from animal blood, such as calf serum.

Throughout this specification, unless the context requires otherwise, the words “comprise,” “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory but that no other elements are optional and may or may not be present depending upon whether or not they affect the activity or action of the listed elements.

The term “individual,” as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adults and juveniles (i.e., children) and infants. It is not intended that the term “individual” connotes a need for medical treatment, therefore, an individual may voluntarily or involuntarily be part of experimentation, whether clinical or in support of basic science studies. The term “subject” or “individual” may be used interchangeably and refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “reduce,” “inhibit,” “diminish,” “suppress,” “decrease,” “prevent,” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including a reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition but an improvement in the outlook of a disease or condition. In specific embodiments, treatment refers to the lessening in severity or extent of at least one symptom and may alternatively or in addition refer to a delay in the onset of at least one symptom.

The term “organoid” as used herein refers to self-organized, three-dimensional tissue cultures that are derived from cells that have self-renewal and differentiation capacities. In specific cases, the term applies to organoids having specific shapes or substantially similar to specific shapes, such as spheroids. In specific cases, the cells that have self-renewal and differentiation capacities are fibroblasts.

The term “fibroblast spheroid-derived materials” as used herein refers to exosomes, cells separating from the spheroids, lysate, conditioned media, and/or apoptotic bodies, etc.

The term “tackifier” as used herein refers to a bio-safe agent that may be included in a liquid medium that increases the viscosity of the medium and in a specific embodiment preserves the fibroblast spheroids in a liquid matrix.

### II. General Embodiments

Embodiments of the disclosure concern methods and compositions for preparation of 3D cellular structures, such as organoids of any shape that allow for the structures to be utilized as extended time release agents and/or also that allow for improved handling of the structures, such as for transport. Embodiments of the disclosure may use one or a combination of the methods for preparation, compositions for preparation, methods of transport, and/or methods of use of the organoids. The organoids are produced such that they may initiate or maintain a desired activity at a target destination, such as at a desired tissue and/or organ in vivo, at a desired culture vessel, at a research facility, at a clinical facility, or a combination thereof. Such activity may be of any kind, such for research and/or therapeutic purposes. The activity may encompass improved viability and/or biological activity following transport and/or the ability to provide extended time release of one or more particular agents (including cells themselves and/or agents produced therefrom). Examples of research applications include drug testing, organ development analysis, surrogate for animal testing, tissue regeneration, bio-assay development, organoid formation testing, pharmaceutical testing, biological response testing, etc.

In specific embodiments, the organoids are produced such that they may (1) initiate or maintain extended time release immune modulation, (2) initiate or maintain extended time release of activated or inactivated fibroblasts and/or fibroblast-derived material, and/or (3) initiate or maintain extended time release migration and proliferation to a targeted tissue or organ for any purpose, including for repair, regeneration and/or reversal of involution. The cell structure of the organoid, as opposed to a plurality of single cells, allows for enhanced cell contact inhibition, the fibroblast cells are naturally reduced in their proliferation rate and metabolism rate to minimize oxygen consumption and nutrient consumption.

A. Embodiments of Production of the Organoids

In various embodiments, the methods and compositions of the present disclosure provide particular actions for preserving and transporting fibroblasts in the form of organoids, including under ambient conditions, for example, and efficiently ensuring their survival rate and biological activity. In specific embodiments, fibroblast spheroids are generated, such as spontaneously in some cases. They may be suspended in a liquid culture medium that ensures basic energy needs, nutrient maintenance needs, and appropriate pH, e.g., that allows for the reduction of shear stress damage caused during transportation. Production of the organoids allows for long-distance transportation needs, including under ambient conditions and/or allows for loading of cell products in plastic, glass, metal, and/or rubber containers, such as for high-density and large-scale delivery. In some cases, following arrival at the intended destination, one may or may not remove the liquid matrix, such as with a centrifuge or by any other means. The organoids may be stored prior to removal of the media. In particular embodiments, the collected cells have clinically beneficial viability and full biological function within 0-21, 0-15, 0-10, 0-5, 1-21, 1-15, 1-10, 1-5, 5-21, 5-15, 5-10, 10-21, 10-15, or 15-21 days, including any value or range derivable therein. As such, the cells may be utilized within 0-21, 0-15, 0-10, 0-5, 1-21, 1-15, 1-10, 1-5, 5-21, 5-15, 5-10, 10-21, 10-15, or 15-21 days, including any value or range derivable therein, upon their production or arrival of their destination. The cells may be utilized immediately or within 0, 1, 2, 3, 4, 5, 6, 7, 8, 9, 10, 11, 12, 13, 14, 15, 16, 17, 18, 19, 20, 21, or more days, including any value or range derivable therein, upon their production or arrival of their destination. A destination may be for clinical use, clinical trial use, and/or research and development use, in specific embodiments.

As an initial step of the methods, or soon after an initial step of the methods, fibroblast cells are manipulated to form organoids (including spheroids) under appropriate suspension culture conditions. In some embodiments for fibroblast spheroid formation, a U/V bottom type ultra-low attachment culture plate or hanging drop method can be used, according to different conditions. For example, cell organoids of different sizes may be generated according to the density of fibroblasts in the medium. In some cases, surface tension on a substrate is manipulated to enhance formation of the organoids. In a specific case, a surface is treated with a hydrophobic material except for a hydrophilic area upon which individual cells or clumps of cells are placed to facilitate production of the organoid. In some cases, spheroids can be formed by forming a core-shell structure within a pipette tip (e.g., (1) making the core spheroid in a single pipette tip and culturing for 48 hours; (2) creating a second layer on the spheroids with cells in the same pipette tip and culturing for 48 hours, repeating as desired to add additional layers of cells and/or cell types to the spheroid; and (3) after the cells spheroids form in the pipette tip for 96 hours, collecting the cell spheroids from the pipette tip by ejecting the liquid containing the spheroid from inside the pipette tip, removing the liquid matrix using centrifugation or filtration, and collecting the cell spheroids). It is also possible to directly use ultra-low attachment culture plates or glass culture flasks to spread fibroblasts into vessels or culture dishes at high density with low-speed stirring to form clumps of different sizes spontaneously.

In particular embodiments, the size of the spheres can range from 50 microns to 500 microns, although small and larger sizes may also be used. In particular embodiments, the sphere size can be between about 50-500, 50-400, 50-300, 50-200, 50-100, 100-500, 100-400, 100-300, 100-200, 200-500, 200-400, 200-300, 300-500, 300-400, or 400-500 microns, including any value or range derivable therein. The sphere size may be about 50, 75, 100, 125, 150, 175, 200, 225, 250, 275, 300, 325, 350, 375, 400, 425, 450, 475, or 500 microns, including any value or range derivable therein. In specific cases, the size of the organoid is generally controlled by the duration of time of culture and the culture media, optionally among other culture parameters.

In particular embodiments, the organoids comprise a particular range of cell numbers. In specific cases, the number of cells in the organoid are generally controlled by the duration of time of culture and the culture media, optionally among other culture parameters. In specific embodiments, the organoids comprise at least about or no more than about 10³, 10⁴, 10⁵, 10⁶, 10⁷, 10⁸, 10⁹, or 10¹⁰ cells per organoid, including any value or range derivable therein.

In some embodiments, fibroblast spheroids are generated upon culture in an incubator at about 37° C., such as for about 24-48 hours. The incubator may or may not be set at about 5% carbon dioxide and greater than about 80% humidity. Stem cell clumps can also form spontaneously at 20-37° C., although other conditions may be utilized therewith, in some cases.

In particular embodiments, for formation the fibroblast cell spheroids may be cultured for 24-48, 24-44, 24-40, 24-36, 24-30, 24-28, 28-48, 28-44, 28-40, 28-36, 28-30, 30-48, 30-44, 30-40, 30-36, 36-48, 36-44, 36-40, 40-48, 40-44, or 44-48 hours, including any value or range derivable therein, in an incubator of suitable conditions. In specific embodiments, the incubator may be set at a particular temperature, such as at about 20, 21, 22, 23, 24, 25, 26, 27, 28, 29, 30, 31, 32, 33, 34, 35, 36, or 37° C., including any value or range derivable therein. A range of temperatures includes about 20-37, 20-35, 20-30, 20-27, 20-25, 20-22, 22-37, 22-35, 22-33, 22-30, 22-27, 22-25, 25-37, 25-35, 25-33, 25-30, 25-27, 27-37, 27-35, 27-33, 27-30, 30-37, 30-35, 30-33, 33-37, 33-35, or 35-37° C., including any value or range derivable therein.

In specific embodiments, the incubator may utilize a certain percentage of a gas, such as carbon dioxide, oxygen, and/or nitrogen. The carbon dioxide may be at 3, 4, 5, 6, 7, or 8%. Ranges of carbon dioxide include about 3-8, 3-7, 3-6, 3-5, 3-4, 4-8, 4-7, 4-6, 4-5, 5-8, 5-7, 5-6, 6-8, 6-7, or 7-8%, including any value or range derivable therein. In specific embodiments, the incubator may be set at a particular percentage of humidity, such as about 65, 70, 75, 80, 85, or 90% humidity, including any value or range derivable therein. The oxygen in the incubator may be 0.5-25% oxygen, including 0.5-25, 0.5-20, 0.5-15, 0.5-10, 0.5-5, 0.5-1, 1-25, 1-20, 1-15, 1-10, 1-5, 5-25, 5-20, 5-15, 5-10, 10-25, 10-20, 10-15, 15-25, 15-20, or 20-25%, including any value or range derivable therein. The oxygen in the incubator may be 0.5, 1, 2, 3, 4, 5, 6, 7, 8, 9, 10, 11, 12, 13, 14, 15, 16, 17, 18, 19, 20, 21, 22, 23, 24, or 25%, including any value or range derivable therein. The nitrogen in the incubator may be 1-75% nitrogen, including 1-75, 1-70, 1-60, 1-50, 1-40, 1-30, 1-20, 1-10, 1-5, 5-75, 5-70, 5-60, 5-50, 5-40, 5-30, 5-20, 5-10, 10-75, 10-70, 10-60, 10-50, 10-40, 10-30, 10-20, 20-75, 20-70, 20-60, 20-50, 20-40, 20-30, 30-75, 30-70, 30-60, 30-50, 30-40, 40-75, 40-70, 40-60, 40-50, 50-75, 50-70, 50-60, 60-75, 60-70, or 70-75% nitrogen, including any value or range derivable therein. The nitrogen in the incubator may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75% nitrogen, including any value or range derivable therein.

A range of humidities may be utilized, such as about 65-90, 65-85, 65-80, 65-75, 65-70, 70-90, 70-85, 70-80, 70-75, 75-90, 75-85, 75-80, 80-90, 80-85, or 85-90%, including any value or range derivable therein. Other incubator conditions and culture durations may be present or required.

In various embodiments, the size of the produced spheroid is able to be controlled at least in part. The desired size may be dictated for one or more purposes, such as a desired size for production purposes, a desired size for transportation purposes, and/or a desired size for a research and/or therapeutic application. In some embodiments, a plurality of organoids to be utilized comprise substantially the same size. In some embodiments, a first plurality of organoids may be combined with a second plurality of organoids of a different size before transportation and/or research and/or therapeutic application; in such cases, the first and second pluralities may have been produced under different conditions, such as different culture parameters, including different durations of culture time. The produced size of the organoids may be dictated by adjusting the concentration of fibroblast cells used to initiate spheroid formation. Such adjusting of the concentration may or may not occur by dilution; by the selection of the base media; by the selection of one or more additives to the media (such as sugar, serum, non-essential amino acids, L-glutamine, a combination thereof, etc.); by selection of the amount of carbon dioxide in the incubator; by the temperature in the incubator; by the duration of time of production of the spheroid; using an outside source of a gas or gas mixtures to feed the incubator; or a combination thereof. The concentration may be increased for certain purposes and decreased for certain purposes. The adjustment of the concentration may increase the size of the organoid by an approximate certain fold level, such as about 2-fold, 5-fold, 10-fold, 100-fold, 1000-fold, 10,000-fold, etc., including any value or range derivable therein. The adjustment of the concentration may decrease the size of the organoid by an approximate certain fold level, such as about 2-fold, 5-fold, 10-fold, 100-fold, 1000-fold, 10,000-fold, etc., including any value or range derivable therein.

In some embodiments, the size of the organoids can be modified to accommodate varying clinical needs (also see below), such as adjusting the concentration of the cells and/or adjusting the duration of time of growth of the organoid. For example, in embodiments wherein a tissue is in need of repair, regeneration and/or reversal of involution, the size of the organoid may be particularly produced, depending on the tissue site in need of the organoids. In some embodiments, the organoid is utilized as a therapeutic delivery agent wherein the therapeutic(s) delivered by the organoid is the fibroblasts themselves and/or one or more agents derived from the fibroblasts. In such cases, the organoid may act as an extended time release mechanism that allows for the release of the therapeutic(s) over a desired period of time. The desired period of time may be when there is a detectable improvement at the site (either by visual or assaying means). The desired period of time may be when there is complete healing at the site (either by visual or assaying means). In some cases, the desired period of time may be the time to be able to initiate or maintain immune modulation as a result of the extended time release. In some cases, the desired period of time may be the time to be able to initiate or maintain activated or inactivated fibroblasts and/or fibroblast-derived material upon delivery and as a result of the extended time release. In some cases, the desired period of time may be the time to initiate or maintain migration and/or proliferation to a targeted tissue or organ for any purpose (including for any purpose, such as repair, regeneration and/or reversal of involution) and as a result of the extended time release. In some cases, the desired period of time may be the time to repair, regenerate and/or reverse involution.

In some embodiments, the organoids following production are stored under suitable conditions. In at least specific cases, the temperature of the storage environment is about 2° C. to about 37° C. In specific embodiments, the storage environment is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37° C., including any value or range derivable therein. The storage temperature may be in range of suitable temperatures, including at least about 2-37, 2-35, 2-30, 2-25, 2-20, 2-15, 2-10, 2-5, 5-37, 5-35, 5-30, 5-25, 5-20, 5-15, 5-10, 10-37, 10-35, 10-30, 10-25, 10-20, 20-37, 20-35, 20-30, 20-25, 25-37, 25-35, 25-30, 30-37, 30-35, or 35-37° C., including any value or range derivable therein.

B. Embodiments of Transportation and/or Therapeutic Use of the Organoids

Once the organoids are generated, they may be stored and/or they may be transported to an intended destination. The organoids may be suspended in a liquid base medium that includes one or more tackifiers to produce a liquid storage matrix. The liquid portion of the liquid storage matrix may comprise any base cell culture medium compatible with fibroblasts, such as one containing basic nutrients and an acid-base balance system; the components may comprise low-glucose medium, such as DMEM low-glucose medium and/or another medium containing 1 mM to 20 mM (e.g., 5 mM) glucose; anywhere from about 0 to 20% human serum; about 0 to 5% non-essential amino acids; and/or about 0 to 5% L-glutamine. The tackifier may be a food-grade additive that can increase the viscosity of the liquid matrix, such as Methylcellulose or any other agent capable of increasing viscosity, and may be at a specific concentration of anywhere from about 0 to 5%. The base medium may contain other materials and/or conditions to facilitate culturing, such as growth factors, sugars, and/or amino acids.

In embodiments wherein the liquid storage matrix for storage and/or transportation may comprise serum, including human serum, the amount may be about 0-20, 0-15, 0-10, 0-5, 1-20, 1-15, 1-10, 1-5, 5-20, 5-15, 5-10, 10-20, 10-15, or 15-20% human serum, including any value or range derivable therein. The amount of serum may be about 0, 1, 2, 3, 4, 5, 6, 7, 8, 9, 10, 11, 12, 13, 14, 15, 16, 17, 18, 19, or 20%, including any value or range derivable therein.

In embodiments wherein the media for storage and/or transportation may comprise non-essential amino acids, the amount may be about 0-5, 0-4, 0-3, 0-2, 0-1, 1-5, 1-4, 1-3, 1-2, 2-5, 2-4, 2-3, 3-5, 3-4, or 4-5% non-essential amino acids, including any value or range derivable therein. The amount of non-essential amino acids may be about 0, 1, 2, 3, 4, or 5% non-essential amino acids, including any value or range derivable therein. The non-essential amino acids may include glycine, L-alanine, L-aspargine monohydrate, L-aspartic acid, L-glutamic acid, L-proline, L-serine, L-histidine, Isoleucine, L-lysine hydrochloride, L-serine, L-tryptophan, and/or L-valine.

In embodiments wherein the liquid storage matrix for storage and/or transportation may comprise about 0 to 5% L-glutamine, the amount may be 0-5, 0-4, 0-3, 0-2, 0-1, 1-5, 1-4, 1-3, 1-2, 2-5, 2-4, 2-3, 3-5, 3-4, or 4-5% L-glutamine, including any value or range derivable therein. The amount of L-glutamine may be about 0, 1, 2, 3, 4, or 5% L-glutamine, including any value or range derivable therein.

In embodiments wherein the liquid storage matrix for storage and/or transportation may comprise one or more tackifiers, the amount may be 0-5, 0-4, 0-3, 0-2, 0-1, 1-5, 1-4, 1-3, 1-2, 2-5, 2-4, 2-3, 3-5, 3-4, or 4-5% tackifiers, including any value or range derivable therein. In embodiments wherein the media for storage and/or transportation may comprise one or more tackifiers, the amount of tackifier may be about 0, 1, 2, 3, 4, or 5%, including any value or range derivable therein. In cases wherein two or more tackifiers are used, their sum amount may or may not be about 0-5, 0-4, 0-3, 0-2, 0-1, 1-5, 1-4, 1-3, 1-2, 2-5, 2-4, 2-3, 3-5, 3-4, or 4-5%, including about 0, 1, 2, 3, 4, or 5%, including any value or range derivable therein.

In some embodiments, upon generation the fibroblast spheroids are housed in a suitable container. The range of spheroids per container may vary according to need, but in specific cases the range is 1-10 million cells per milliliter of preservation liquid matrix. In specific embodiments, the packaging and preservation of the fibroblast spheroids are in a sealed container, including a sterile one. In specific embodiments, the container is a plastic container, such as one made from chemically inert materials including polyethylene, polypropylene, and melamine. The container may also be constructed of other materials that would not adversely impact the cells, such as glass, coated metal, or other polymers such as polystyrene, branched polymer hydrogels, and cycle-olefin polymers.

Embodiments of the present disclosure allow for the spheroids to be transported under ambient conditions, and this may be before or after storage. In specific embodiments, the spheroids are transported in a temperature range from 2-35° C. The spheroids may be transported in a temperature range of 2-35, 2-30, 2-25, 2-20, 2-15, 2-10, 2-5, 5-35, 5-30, 5-25, 5-20, 5-15, 5-10, 10-30, 10-25, 10-20, 10-15, 15-35, 15-30, 15-25, 15-20, 20-35, 20-30, 20-25, 25-35, 25-30, or 30-35° C., including any value or range derivable therein. The spheroids may be transported at 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C., including any value or range derivable therein. In specific embodiments, the temperature for transport is not more than about 38° C. and/or is not less than 0° C., although in other embodiments the spheroids are transported cryogenically.

In specific embodiments, the cell container is not subject to intense energy or light waves, including intense light irradiation, X rays, such as is used at airports, or any intense light source during at least part if not all of transportation. In specific cases, the cell container and/or the incubator during preparation is not subject to light during at least part if not all of transportation.

The produced organoids may be transported and/or used within about 0-50 days following production. In some cases, the organoids are transported and/or used on the same day as production, within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to 50 days, including any value or range derivable therein. In some embodiments, the organoids are transported and/or used within 1-50, 1-40, 1-30, 1-25, 1-20, 1-10, 1-5, 5-50, 5-40, 5-30, 5-25, 5-20, 5-10, 10-50, 10-40, 10-30, 10-25, 10-20, 20-50, 20-40, 20-30, 20-25, 25-50, 25-40, 25-30, 30-50, 30-40, or 40-50 days, including any value or range derivable therein.

In particular embodiment, the fibroblast spheroids may be manipulated such that at least some of the cells are dissociated from the organoid, thereby allowing the dissociated fibroblast cells to restore proliferation or to proliferate at a greater rate than in the organoid. In specific embodiments, the organoids are exposed to one or more proteases, mechanical disruption (such as by pipetting up and down), and/or use of sonication for dissociation. In specific cases, the amount of trypsin is about 0.1-0.3% trypsin, such as about 0.25% trypsin. In particular cases, the spheroids are subjected to about 0.25% trypsin and may be placed in a 37° C. incubator to restore proliferation of the dissociated fibroblasts. In addition, the fibroblasts can be directly plated back into the vessel it was transported in and cultured in normothermia.

In particular embodiments, a plurality of fibroblasts can be released from the spheroids. The rate of release can vary based on size of the spheroids, content of spheroid, how the spheroids were activated, and/or the environment in which it was administered. In particular embodiments, a plurality of fibroblasts can be released from the spheroids and migrate to a desired site (such as a specific organ or tissue). The ability of the released fibroblasts to migrate to a desired site may be because of one or more natural characteristics of the released fibroblasts or because of one or more acquired characteristics of the released fibroblasts, such as characteristics imparted to the fibroblasts by the hand of man. In specific embodiments, the released fibroblasts migrate to a desired site because the cells have become activated by activated or engineered surface markers, nucleic acid modification, and/or expression or excretion of one or more chemokines, one or more cytokines, exosomes, and/or one or more growth factors. In particular embodiments, a plurality of fibroblasts can be released from the spheroids and migrate to a desired site and grow adherently from the spheroids. In specific embodiments, when the spheroids bind to the desired surface, the cells then migrate out of the spheroids onto that desired area and may proliferate. Furthermore, in some embodiments, the fibroblast spheroids can separate from the preservation liquid matrix and directly applied to transplantation site for tissue regeneration or immunotherapy.

In some embodiments, the size of the organoids can be modified to accommodate varying clinical needs, such as adjusting the concentration of the cells and/or adjusting the duration of time of growth of the organoid. For example, in embodiments wherein a tissue is in need of repair, regeneration and/or reversal of involution, the size of the organoid may be particular, depending on the tissue site in need of the organoids. In some embodiments, a specific size of organoid is desired to be able to be injected from a needle. In some embodiments, specific size of organoid is desired to be able to be infused to a specific site in which the vein/artery size.

In some embodiments, the organoid is utilized as a therapeutic delivery agent wherein the therapeutic(s) delivered by the organoid is the fibroblasts themselves and/or one or more agents derived from the fibroblasts. In such cases, the organoid may act as an extended time release mechanism that allows for the release of the therapeutic(s) over a desired period of time. The desired period of time may be when there is a detectable improvement at the site (either by visual or assaying means). The desired period of time may be when there is complete healing at the site (either by visual or assaying means). In some cases, the desired period of time may be the time to be able to initiate or maintain immune modulation as a result of the extended time release. In some cases, the desired period of time may be the time to be able to initiate or maintain activated or inactivated fibroblasts and/or fibroblast-derived material upon delivery and as a result of the extended time release. In some cases, the desired period of time may be the time to initiate or maintain migration and/or proliferation to a targeted tissue or organ for any purpose (including for any purpose, such as repair, regeneration and/or reversal of involution) and as a result of the extended time release. In some cases, the desired period of time may be the time to repair, regenerate and/or reverse involution at a desired site.

In particular embodiments, one can modulate the size of the fibroblast spheroids to adjust the extent and duration of the extended-release function of single fibroblast cells from the organoids and or fibroblast-derived materials in vivo. Such modulation may encompass producing larger-sized organoids for longer extended release of the cells and/or fibroblast-derived materials vs. producing smaller-sized organoids for extended release of the cells and/or fibroblast-derived materials that are not needed for so long. For example, for organ involution, in specific embodiments one may produce organoids that are larger in size than for localized tissue repair.

In embodiments of the disclosure, one may utilize the organoid fibroblasts as a method of “extended time release” of the fibroblast cells and/or fibroblast cell-derived or excreted materials. Such agents may be used for any suitable purpose, including for the purpose of immune modulation, chronic disease treatment, tissue repair, tissue regeneration, targeting of specific tissue for migration, and/or proliferation for subsequent growth, and/or differentiation.

In specific embodiments, the fibroblasts from the organoids and/or fibroblast-derived materials from the organoids may be released slowly over a period of time from the spheroid to elicit an immune modulation response or other biological responses that is therapeutic for the treatment of one or more chronic diseases. For immune modulation, the organoids may be used for autoimmune disorders, such as MS, and in skin disorders such as eczema or psoriasis that have an immune disregulation component. The organoids may be used in treatment of cancer of any kind, given that cancer cells use immune regulation to hide from an immune response. The organoids may be used as a biologic time release in the thymus and spleen.

In particular embodiments, the fibroblast organoids are administered to an individual in need thereof by any suitable administration, including one that is selected for the application being treated. Such administrations may be local or systemic and may include injection, infusion, spray, and/or inclusion in a 3D matrix. In specific embodiments, there is one administration to the individual or multiple administrations. In cases wherein there are multiple administrations, the duration in time between the administrations may be within 1, 2, 3, 4, 5, 6, 7, 8, 9, 10, 11, or 12 hours, or within 1, 2, 3, 4, 5, 6, or 7 days, or within 1, 2, 3, or 4 weeks, or within 1, 2, 3, 4, 5, 6, 7, 8, 9, 10, 11, or 12 months, or within 1, 2, 3, 4, 5, or more years between administrations, including any value or range derivable therein.

In specific embodiments, the spheroids are delivered to or onto a damaged tissue; in specific embodiments thereafter, they migrate and/or proliferate to a desired site, such as to regenerate the damaged tissue through differentiation and/or stem cell recruitment.

In certain embodiments, the organoid fibroblasts have been activated, such as to express one or more surface molecules that target a specific tissue or organ for migration and proliferation for tissue repair and/or eliciting an immune response at the target site. The fibroblasts may be activated by any suitable means, such as through one or more chemical agents (including small molecules), RNA, micro RNA, RNAi, DNA, viral nucleic acid, and/or exosomes. Specific examples include 6SM, p53 peptide, I09/RG7388, NUT/nutlin-3a, IQK, 41P, 2FS, TX6/TX64014, Estradiol, 4-Hydroxytamoxifen, Bazedoxifene, AZD9496, Testosterone, Bicalutamide, PT2399, ZTD, VH298, T28, OWC, 76Z, JQ1, or a combination thereof.

### III. Embodiments of Research Purposes

In some embodiments, the produced organoids are utilized for research purposes of any kind. Organoids for research use are particularly well-suited, given that they are self-organizing, 3D culture systems that are highly similar to actual human organs and, in some cases, are histologically indistinguishable from human organs. The organoids can be utilized to obtain information about mechanisms that underlie human development and organ regeneration, highlighting their value for basic biological research in addition to their use for pharmaceutical drug testing and molecular medicine.

The organoids may be commercially sold for research purposes. Produced organoids may be transported by methods and compositions encompassed herein to research institutions for any purpose. In specific embodiments, the organoids are used as an alternative to animal models for pre-clinical drug testing. The application of these organoids may be to explore organ function and to examine approaches for drug development.

The organoids of the disclosure may be utilized to study infectious diseases (including viral, bacterial, fungal, protozoan, etc.), genetic disorders, and cancers, as specific examples.

The source of the fibroblasts for the organoids for research purposes may be from the genetic engineering of the fibroblasts or when the organoids are generated from patient biopsy samples. Therefore, the production of the organoids (including the type of fibroblasts, their optional modification/activation, the size of the organoid, etc.) may be tailored to the specific needs of a researcher or research institution (whether private or public). The organoids may be produced to characterize biological processes that are specific to the human body and cannot be modelled in other animals, for example.

### IV. Kits of the Disclosure

Any of the compositions described herein may be comprised in a kit. In a non-limiting example, fibroblasts, tissues that comprise fibroblasts, organoids comprising fibroblasts, reagents for culturing, one or more tackifiers, or a combination thereof, each in suitable container means. The kit may comprise an organ-on-a-chip system.

The component(s) of the kits may be packaged in a suitable solution, where appropriate. The component(s) of the kits may, in certain embodiments, be either in aqueous media or in lyophilized form. The container means of the kits may generally include at least one vial, test tube, flask, bottle, syringe or other container means, into which a component may be placed, and particularly, suitably aliquoted. Where there is more than one component in the kit, the kit also may generally contain a second, third or other additional container into which the additional component(s) may be separately placed. However, various combinations of components may be comprised in a vessel, such as a vial. The kits of the present disclosure also will typically include a means for containing the components in close confinement for commercial sale. Such containers may include injection or blow molded plastic containers into which the desired vessels are retained, for example.

Irrespective of the number and/or type of containers, the kits of the disclosure may also comprise, and/or be packaged with, an instrument for assisting with the injection/administration and/or placement of the organoids within the body of an animal. Such an instrument may be a syringe, pipette, forceps, and/or any such medically approved delivery vehicle.

### EXAMPLES

### I. Example 1—Generation of Spheroids from Mouse Fibroblasts

Mouse skin fibroblasts have high viability after being stored at room temperature for 7 days.

One strain of mouse fibroblast cells was isolated from mouse skin fibroblasts. For validation experiments, cells were grown in a normal cell culture environment (e.g., 37 degrees Celsius, 5% carbon dioxide, 90% humidity) to 80% confluency for experiments. Examples of experimental steps are as follows:
1. Isolate the cells, remove the medium, and wash twice with 1×PBS.
2. Dissociate cells with 0.25% trypsin for 3 minutes.
3. Suspend cells in a suitable medium (e.g., that comprises low glucose, human serum, non-essential amino acids, and/or L-glutamine, in specific embodiments) to collect the cells.
4. Adjust the cell concentration to 8×105 cells/mL and use the hanging drop method to prepare stem cell spheres, at 25 uL per drop.
5. Place the culture plate into the normal cell culture environment for 48 hours.
6. Collect the stem cell pellets from the hanging drop.
7. Centrifuge the stem cell pellets at 1000 rpm for 3 minutes and remove the supernatant.
8. Add the preservation matrix to resuspend the cells and adjust the cell density to 5 million cells per mL of the liquid matrix.
9. Add the cells to a 15 mL plastic centrifuge tube, seal the tube with parafilm, and store the tube in the dark at room temperature (e.g. at about 20° C.) for 7 days starting from day 0.
10. On day 7, place the centrifuge tube in the incubator for 2 hours, centrifuge the cells, remove the supernatant, and wash twice with 1×PBS.
11. Dissociate cells with 0.25% trypsin for 3 minutes.
12. Resuspend cells in stem cell medium (e.g. low glucose DMEM with 10% human serum, 1% non-essential amino acids, and/or 5% L-glutamine).
13. The cells can be tested, applied, or returned to the normal culture environment to continue culturing.

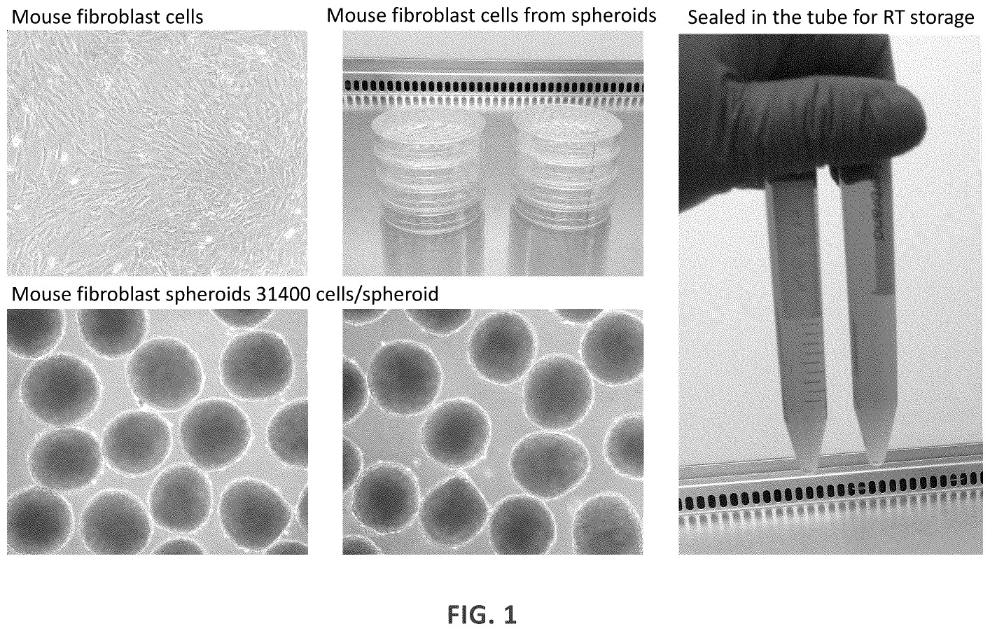

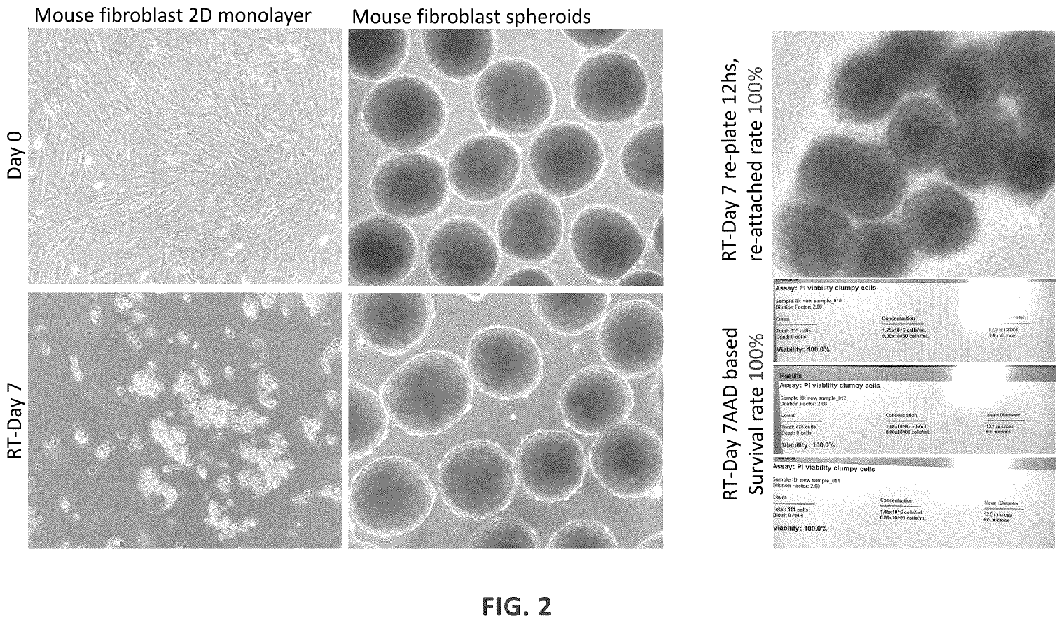

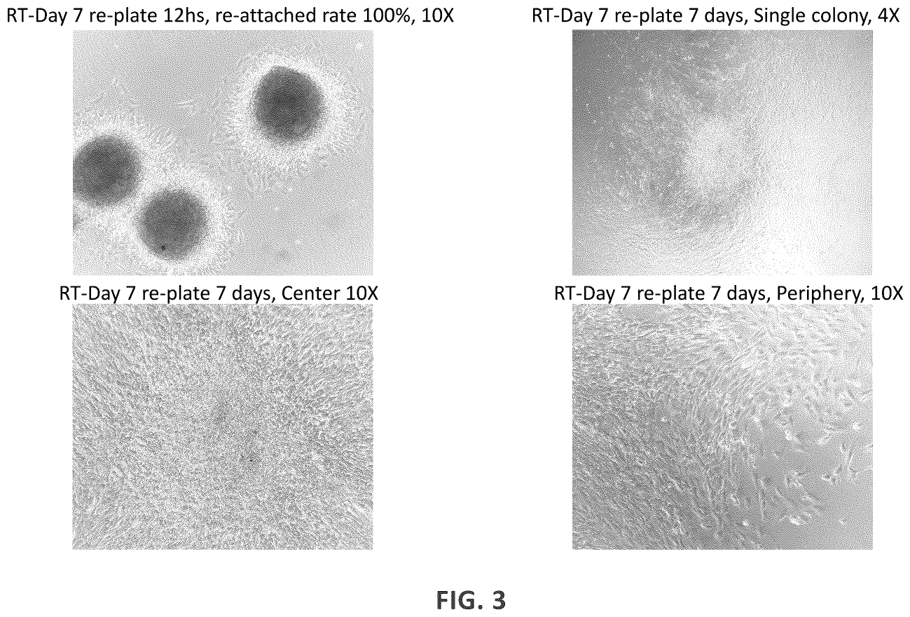

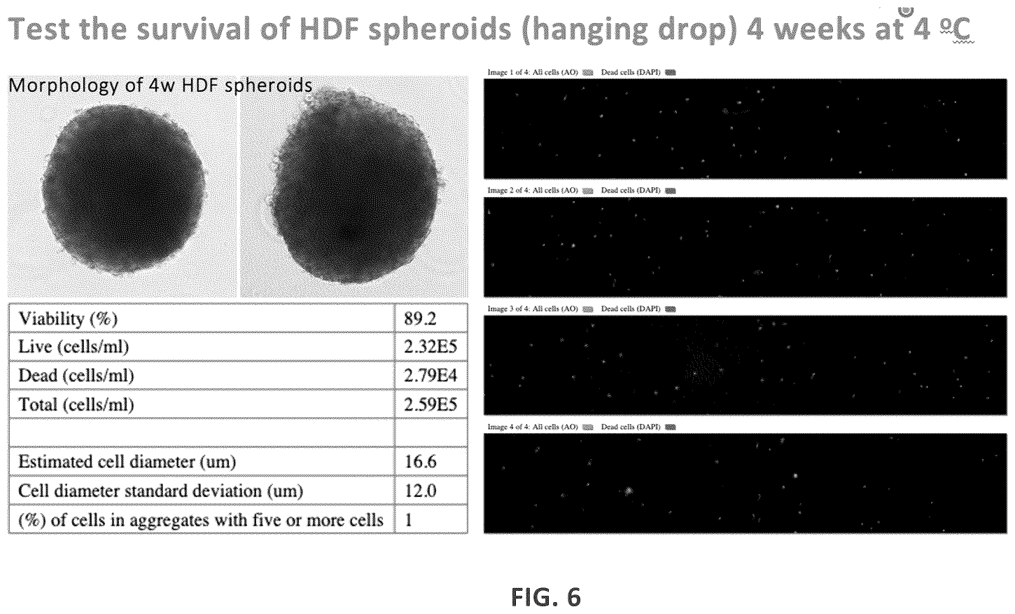

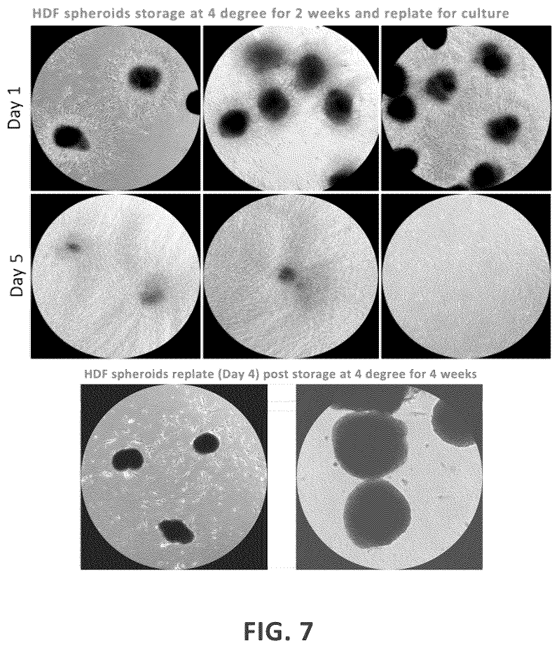

With this method, mouse skin fibroblasts can obtain a 100% survival rate and have normal cell morphology, cell proliferation, and migration ability. (FIGS. 1-8)

### II. Example 2—Generation of Spheroids from Human Fibroblasts

Human fibroblasts were kept at room temperature for 7 days with high viability after pelleting.

One strain of human fibroblast cells was isolated from an individual. The skin fibroblasts were used for validation experiments. Cells were grown in a normal cell culture environment (37 degrees Celsius, 5% carbon dioxide, 90% humidity) to 80% confluency for experiments. Examples of the steps are as follows:
1. Isolate the cells, remove the medium, and wash twice with 1×PBS.
2. Dissociate cells with 0.25% trypsin for 3 minutes.
3. Suspend cells in a suitable medium (e.g., that comprises low glucose, human serum, non-essential amino acids, and/or L-glutamine, in specific embodiments) to collect the cells.
4. Adjust the cell concentration to 8×105 cells/mL and use the hanging drop method to prepare stem cell spheres, at 25 uL per drop.
5. Place the culture plate into the normal cell culture environment for 48 hours.
6. Collect the stem cell pellets from the hanging drop.
7. Centrifuge the stem cell pellets at 1000 rpm for 3 minutes and remove the supernatant.
8. Add the preservation matrix to resuspend the cells and adjust the cell density to 5 million cells per mL of the liquid matrix.
9. Add the cells to a 15 mL plastic centrifuge tube, seal the tube with parafilm, and store the tube in the dark at room temperature (e.g., at about 20° C.) for 7 days starting from day 0.
10. On day 7, place the centrifuge tube in the incubator for 2 hours, centrifuge the cells, remove the supernatant, and wash twice with 1×PBS.
11. Dissociate cells with 0.25% trypsin for 3 minutes.
12. Resuspend cells in stem cell medium (e.g., low glucose DMEM with 10% human serum, 1% non-essential amino acids, and/or 5% L-glutamine).
13. The cells can be tested, applied, or returned to the normal culture environment to continue culturing.

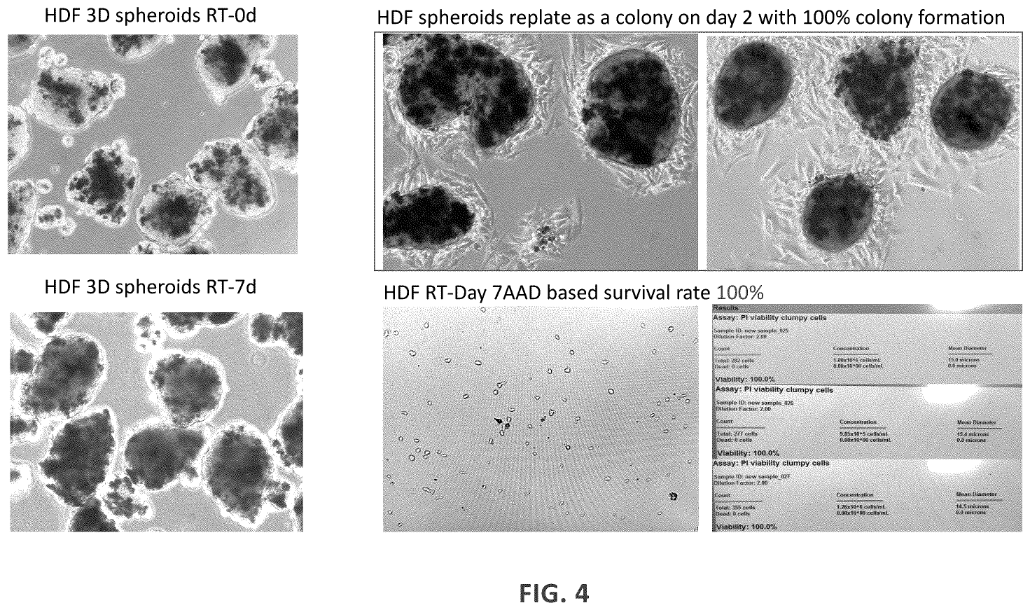

This method allows human dermal fibroblasts to obtain a 100% survival rate and have normal cell morphology, cell proliferation, and migration ability. (FIG. 4)

### III. Example 3—Generation of Spheroids from Human Fibroblasts

Human dermal fibroblast (HDF) spheroids can survive well and form the colonies for up to 4 weeks of storage at 4° C.

One strain of human fibroblast cells was isolated from an individual. The skin fibroblasts were used for validation experiments. Cells were grown in a normal cell culture environment (e.g., at 37° C., 5% carbon dioxide, 90% humidity) to 80% confluency for experiments. Examples of the steps are as follows:
1. Isolate the cells, remove the medium, and wash twice with 1×PBS.
2. Dissociate cells with 0.25% trypsin for 3 minutes.
3. Suspend cells in a suitable medium (e.g., that comprises low glucose, human serum, non-essential amino acids, and/or L-glutamine, in specific embodiments) to collect the cells.
4. Adjust the cell concentration to 8×105 cells/mL and use the hanging drop method to prepare stem cell spheres, at 25 uL per drop.
5. Place the culture plate into the normal cell culture environment for 48 hours.
6. Collect the stem cell pellets from the hanging drop.
7. Centrifuge the stem cell pellets at 1000 rpm for 3 minutes and remove the supernatant.
8. Add the preservation matrix to resuspend the cells and adjust the cell density to 5 million cells per mL of the liquid matrix.
9. Add the cells to a 15 mL plastic centrifuge tube, seal the tube with parafilm, and store the tube in the dark at room temperature (e.g., at about 20° C.) for 14 days starting from day 0.
10. On day 14, place the centrifuge tube in the incubator for 2 hours, centrifuge the cells, remove the supernatant, and wash twice with 1×PBS.
11. Dissociate cells with 0.25% trypsin for 3 minutes.
12. Resuspend cells in stem cell medium (e.g., low glucose DMEM with 10% human serum, 1% non-essential amino acids, and/or 5% L-glutamine).
13. The cells can be tested, applied, or returned to the normal culture environment to continue culturing.

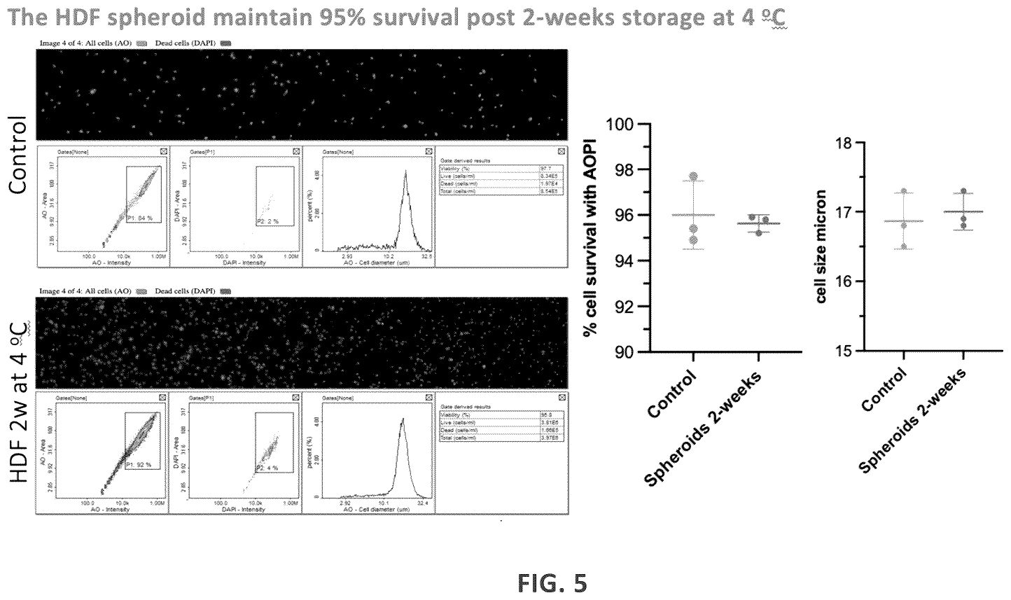

This method allows human dermal fibroblasts to obtain an 89-95% survival rate and have normal cell morphology, cell proliferation, and migration ability. (FIGS. 5-7)

### IV. Example 4—Validation of Spheroids from Human Fibroblasts

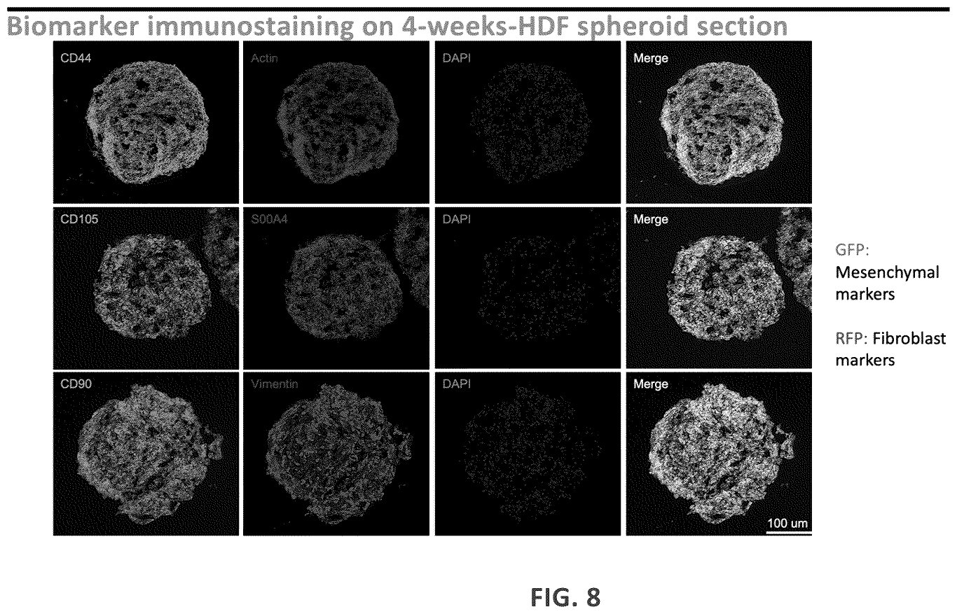

The HDF spheroids can survive well and form colonies for up to 4 weeks of storage at 4° C. The HDFs in the spheroids maintain the dermal fibroblast phenotype. (FIG. 8)

Cryosections can be used to validate that HDFs in spheroids maintain the skin fibroblast phenotype. Spheroid sections that are 10 um thick can be stained with anti-CD90 antibodies to detect mesenchymal stem cells and with Vimentin to detect HDFs. The nuclei can be stained with DAPI nuclear stain. 1% fetal serum in 1×DPBS can be used as blocking buffer, incubation buffer, and mounting medium. Examples of the steps are as follows:
1. Allow cryo-tissue sections to air-dry sections briefly at room temperature (RT).
2. Surround the sections with a hydrophobic pen.
3. Rehydrate the sections twice for 10 minutes in 1×DPBS in staining dishes.
4. Add blocking buffer and block for 1 hour at RT in a wet chamber.
5. Remove the blocking buffer and add incubation buffer with the primary antibody.
6. Incubate the slides with the primary antibody overnight at 4° C. in a wet chamber.
Important: Some antibodies require incubation at RT. Please refer to the corresponding antibody data sheet.
7. Wash the slides three times for 10 minutes in 1×DPBS at RT in staining dishes.
8. Transfer the slides to the wet chamber and apply the incubation buffer with the secondary antibody diluted to the manufacturer's recommended concentration.
9. Incubate the slides for 1 hour at RT.
10. Wash slides three times for 10 minutes in 1×DPBS at RT in staining dishes.
11. Optional: Add DAPI solution for 10 minutes in 1×DPBS at RT.
12. Wash slides three times for 10 minutes in 1×DPBS at RT in staining dishes.
13. Remove the hydrophobic circle around the tissue section.
14. Mount slides and microscope.

### REFERENCES

1. Tschumperlin, D. J., Fibroblasts and the ground they walk on. Physiology (Bethesda), 2013. 28(6): p. 380-90.
2. Lee, J. H., Y. S. Han, and S. H. Lee, Long-Duration Three-Dimensional Spheroid Culture Promotes Angiogenic Activities of Adipose-Derived Mesenchymal Stem Cells. Biomol Ther (Seoul), 2016. 24(3): p. 260-7.
3. Shimura, M., et al., Room temperature-induced apoptosis of Jurkat cells sensitive to both caspase-1 and caspase-3 inhibitors. Cancer Lett, 1998. 132(1-2): p. 7-16.
4. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.
5. Yong, K. W., et al., Cryopreservation of Human Mesenchymal Stem Cells for Clinical Applications: Current Methods and Challenges. Biopreserv Biobank, 2015. 13(4): p. 231-9.
6. Jang, T. H., et al., Cryopreservation and its clinical applications. Integr Med Res, 2017. 6(1): p. 12-18.

Although the present disclosure and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of producing fibroblast organoids, the method comprising:
producing a plurality of fibroblast organoids; and
suspending the fibroblast organoids in a storage matrix comprising low-glucose base media; 0 to 20% human serum; 0 to 5% non-essential amino acids; and/or 0 to 5% L-glutamine; and optionally at least one tackifier.

2. The method of claim 1, wherein the storage matrix comprises:
(a) about 0-20, 0-15, 0-10, 0-5, 1-20, 1-15, 1-10, 1-5, 5-20, 5-15, 5-10, 10-20, 10-15, or 15-20% of the human serum;
(b) about 0, 1, 2, 3, 4, 5, 6, 7, 8, 9, 10, 11, 12, 13, 14, 15, 16, 17, 18, 19, or 20% of the human serum;
(c) about 0-5, 0-4, 0-3, 0-2, 0-1, 1-5, 1-4, 1-3, 1-2, 2-5, 2-4, 2-3, 3-5, 3-4, or 4-5% of the non-essential amino acids;
(d) about 0, 1, 2, 3, 4, or 5% of the non-essential amino acids;
(e) about 0-5, 0-4, 0-3, 0-2, 0-1, 1-5, 1-4, 1-3, 1-2, 2-5, 2-4, 2-3, 3-5, 3-4, or 4-5% of the L-glutamine;
(f) about 0, 1, 2, 3, 4, or 5% of the L-glutamine;
(g) about 0-5, 0-4, 0-3, 0-2, 0-1, 1-5, 1-4, 1-3, 1-2, 2-5, 2-4, 2-3, 3-5, 3-4, or 4-5% of the at least one tackifier; and/or
(h) about 0, 1, 2, 3, 4, or 5% of the at least one tackifier.

3. (canceled)

4. (canceled)

5. (canceled)

6. (canceled)

7. (canceled)

8. (canceled)

9. (canceled)

10. The method of claim 1, wherein the at least one tackifier comprises Methylcellulose, Agar, Guar gum, Xanthan gum, Pectin, Collagen, and/or Gelatin.

11. The method of claim 1, wherein:
(a) the fibroblast organoids are about 50-500, 50-400, 50-300, 50-200, 50-100, 100-500, 100-400, 100-300, 100-200, 200-500, 200-400, 200-300, 300-500, 300-400, or 400-500 microns in size;
(b) the fibroblast organoids are about 50, 75, 100, 125, 150, 175, 200, 225, 250, 275, 300, 325, 350, 375, 400, 425, 450, 475, or 500 microns in size;
(c) the fibroblast organoids are produced by:
(1) placing the fibroblasts in a U/V bottom type ultra-low attachment culture plate
(2) the hanging drop method;
(3) applying the fibroblasts to a hydrophilic region of a substrate comprising a substantially hydrophobic surface; and/or
(4) forming a core-shell structure within a pipette tip;
(d) the fibroblast organoids comprise about 10³, 10⁴, 10⁵, 10⁶, 10⁷, 10⁸, 10⁹, or 10¹⁰ fibroblasts per fibroblast organoid.
(e) the fibroblast organoids are produced upon culture in an incubator at about 37° C. for about 24-48 hours, wherein the incubator comprises about 5% carbon dioxide and greater than about 80% humidity;
(f) the fibroblast organoids are produced upon culture for about 24-48, 24-44, 24-40, 24-36, 24-30, 24-28, 28-48, 28-44, 28-40, 28-36, 28-30, 30-48, 30-44, 30-40, 30-36, 36-48, 36-44, 36-40, 40-48, 40-44, or 44-48 hours;
(g) the fibroblast organoids are produced upon culture for about 24, 25, 26, 27, 28, 29, 30, 31, 32, 33, 34, 35, 36, 37, 38, 39, 40, 41, 42, 43, 44, 45, 46, 47, or 48 hours;
(h) the fibroblast organoids are produced upon culture at a temperature of about 20-37, 20-35, 20-30, 20-27, 20-25, 20-22, 22-37, 22-35, 22-33, 22-30, 22-27, 22-25, 25-37, 25-35, 25-33, 25-30, 25-27, 27-37, 27-35, 27-33, 27-30, 30-37, 30-35, 30-33, 33-37, 33-35, or 35-37° C.;
(i) the fibroblast organoids are produced upon culture at a temperature of about 20, 21, 22, 23, 24, 25, 26, 27, 28, 29, 30, 31, 32, 33, 34, 35, 36, or 37° C.;
(j) the fibroblast organoids are produced upon culture in carbon dioxide at about 3-8, 3-7, 3-6, 3-5, 3-4, 4-8, 4-7, 4-6, 4-5, 5-8, 5-7, 5-6, 6-8, 6-7, or 7-8%;
(k) the fibroblast organoids are produced upon culture in carbon dioxide at about 3, 4, 5, 6, 7, or 8%;
(l) the fibroblast organoids are produced upon culture in about 65-90, 65-85, 65-80, 65-75, 65-70, 70-90, 70-85, 70-80, 70-75, 75-90, 75-85, 75-80, 80-90, 80-85, or 85-90% humidity; and/or
(m) the fibroblast organoids are produced upon culture in about 65, 70, 75, 80, 85, or 90% humidity.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The method of claim 1, wherein the producing step comprises modification of one or more parameters to control the size of the produced organoid.

25. The method of claim 24, wherein the one or more parameters comprise fibroblast cell concentration; type and/or amount of base media; type and/or amount of sugar in the base media; type and/or amount of serum in the liquid storage matrix; the type and/or non-essential amino acids; the amount of L-glutamine; the amount of carbon dioxide in an incubator comprising the fibroblast cells; the temperature in the incubator; the duration of time of production of the fibroblast spheroids; or a combination thereof.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 1, wherein the fibroblasts are dissociated from the fibroblast organoid upon being exposed to an effective amount of at least one protease.

41. (canceled)

42. (canceled)

43. The method of claim 1, wherein the fibroblast organoids are used within 0-21 days of suspending step.

44. The method of claim 43, wherein the fibroblast organoids are used in one or more assays or are used for therapy within 0-20, 0-15, 0-10, 0-5, 1-20, 1-15, 1-10, 1-5, 5-20, 5-15, 5-10, 10-20, 10-15, or 15-20 days of the suspending step.

45. The method of claim 44, wherein the fibroblast organoids are used in one or more assays or are used for therapy within 0, 1, 2, 3, 4, 5, 6, 7, 8, 9, 10, 11, 12, 13, 14, 15, 16, 17, 18, 19, or 20 days of the suspending step.

46. The method of claim 44, wherein the fibroblast organoids are used in one or more assays as an alternative to an animal model.

47. The method of claim 44, wherein the assay is for pre-clinical drug testing or drug development, for using the fibroblast organoids to study organ function, and/or to satisfy requirements for drug screening.

48. (canceled)

49. (canceled)

50. The method of claim 1, further comprising the step of exposing the fibroblast organoids to one or more chemicals and/or to one or more drugs.

51. (canceled)

52. The method of claim 1, further comprising the step of administering a therapeutically effective amount of the fibroblast organoids to an individual in need thereof.

53. (canceled)

54. (canceled)

55. (canceled)

56. (canceled)

57. (canceled)

58. (canceled)

59. (canceled)

60. (canceled)

61. The method of claim 52, wherein fibroblasts are dissociated from the fibroblast organoids following administration.

62. The method of claim 61, wherein the fibroblasts are dissociated from the fibroblast organoids as an extended release.

63. (canceled)

64. The method of claim 61, wherein the fibroblasts migrate to a specific tissue or organ.

65. (canceled)

66. The method of claim 64, wherein the specific tissue is connective tissue, epithelial tissue, muscle tissue, and/or nervous tissue.

67. The method of claim 64, wherein the specific organ is the heart, liver, lungs, stomach, spleen, gall bladder, kidney, brain, bladder, and/or intestines.

68. (canceled)

69. (canceled)

70. (canceled)

71. The method of claim 52, wherein the fibroblasts comprise one or more therapeutic agents for release from the fibroblasts.

72. (canceled)

73. (canceled)

74. (canceled)

75. A composition comprising organoids produced by the method of claim 1.

76. (canceled)

77. (canceled)

78. (canceled)

79. (canceled)

80. (canceled)

81. The composition of claim 75, wherein the organoids are in a storage matrix comprising a low-glucose base medium; about 0 to 20% human serum; about 0 to 5% non-essential amino acids; and/or about 0 to 5% L-glutamine; and optionally at least one tackifier.
